(19) United States
(12) Patent Application Publication
(10) Pub. No.: US 2017/0296538 A1
(43) Pub. Date: Oct. 19, 2017

(54) SOLUTE AND SOLUTE MIXTURE, AS WELL AS A COMPOSITION COMPRISING AT LEAST ONE SOLUTE, FOR USE IN THE PREVENTION OR TREATMENT OF COSMETIC OR PATHOLOGIC EFFLORESCENCES CAUSED BY AIRBORNE PARTICLES

(71) Applicant: bitop AG, Witten (DE)

(72) Inventors: Andreas BILSTEIN, Bergheim (DE); Jean KRUTMANN, Düsseldorf (DE)

(21) Appl. No.: 15/513,815

(22) PCT Filed: Sep. 24, 2014
(86) PCT No.: PCT/EP2014/070298

§ 371 Date: Mar. 23, 2017

(30) Foreign Application Priority Data
Sep. 23, 2014 (DE) 10 2014 113 781.7

(51) Int. Cl. A61K 31/505; A61Q 19/04; A61Q 19/02; A61Q 19/08; A61K 8/49

(52) U.S. Cl. CPC A61K 31/505; A61Q 19/08; A61K 8/4953; A61Q 19/02; A61Q 19/04

(57) ABSTRACT

The invention relates to a compatible solute or a solute mixture, as well as to a composition comprising the solute or solute mixture, for use in the prevention or treatment of cosmetic or pathologic efflorescences caused by airborne particles, such as pigmentation accompanying with skin ageing, and de- or hypopigmentation, hyperpigmentation, as well as alterations accompanying with atrophy in the broadest sense. The solute or solute mixture, as well as the composition comprising the solute or solute mixture comprises at least one compound according to formula I, formula II, physiologically compatible salts of formula I and formula II, stereoisomeric forms of the compound of formula I, formula II, and physiologically compatible salts of the stereoisomeric forms, and the solute mixture comprises at least two of the afore-mentioned compounds, wherein in formula I

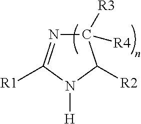

and in formula II

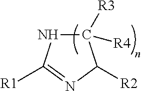

R1=H or alkyl; R2=H, COOH, COO-alkyl or CO—NH—R5; R3 and R4 each independently H or OH; R5=H, alkyl, an amino acid residue, dipeptide residue or tripeptide residue and n=1, 2 or 3 and alkyl corresponds to an alkyl group having C₁-C₄ carbon atoms.

The invention relates to a compatible solute or a solute mixture, as well as to a composition comprising the solute or solute mixture, for use in the prevention or treatment of cosmetic or pathologic efflorescences caused by airborne particles, such as pigmentation accompanying with skin ageing, and de- or hypopigmentation, hyperpigmentation, as well as alterations accompanying with atrophy in the broadest sense. The solute or solute mixture, as well as the composition comprising the solute or solute mixture, comprises at least one compound according to formula I, of formula II, physiologically compatible salts of formula I and formula II, stereoisomeric forms of the compound of formula I, formula II, and physiologically compatible salts of the stereoisomeric forms, and the solute mixture comprises at least two of the afore-mentioned compounds.

Compatible solutes, also referred to as osmolytes, are organic compounds having low molar mass. They are used as protective substances in cosmetic compositions as well as in medical products. For instance as protective substance for the skin, in order to protect the skin cells against dehydration or against damages caused by UV-A- and UV-B-radiation.

Protective effect of the compatible solutes, ectoine and hydroxyectoine, against neurodermatitis is described in DE 10330243 A1. EP 1641442 B1 discloses a protective effect in lung diseases induced by airborne particles.

The exposure of human and animal organism to the influence of airborne particles is a ubiquitous problem. Enormous air pollution of the environment and exposure to airborne particles at the workplace lead to various syndromes. Health-damaging effect of airborne particles has been proved in numerous studies. As a result, numerous norms for the protection of human in environment and at the workplace have been issued. The consequences of exposure to airborne particles exhibit near-term occurring symptoms and diseases, such as, for example, respiratory or cardiovascular diseases. However, the afore-mentioned diseases can also occur only after long-term exposure to airborne particles. It depends on concentration of airborne particles in the air as well as on particle size.

The Word Health Organization has published the norm WHO/SDE/OEH/99.14 for hazard prevention and control of exposure to airborne particles at the workplace. Numerous diseases caused by airborne particles are listed therein, which are evoked by inhalation of airborne particles or by dermal absorption. Another norm ISO4225 specifies sampling and measurement of air quality, and DIN EN 481 comprises a specification and classification of airborne particles and their particle size.

Airborne particles and, in particular, those according to one of the afore-mentioned norms cause skin alterations in human, also referred to as efflorescences. Efflorescences are primarily visible and/or palpable basic elements of a disease of the skin and/or of the subcutis, and comprise primary efflorescences (or primary efflorescences), i.e. efflorescences that has developed from healthy skin without intermediate stage, and secondary efflorescences (or secondary efflorescences), thus efflorescences that has developed from primary efflorescences.

Cosmetic efflorescences, such as, for example, spotted skin in the face or on the hands, have significant influence on aesthetical perception and psychologic constitution of affected human. However, exposure to airborne particles in high concentrations or even also, already at low concentration may cause pathologic efflorescences, for example in the case of immunosuppressive people, people with hypersensitive immune system (e.g. allergy sufferers) or people having a special genetic constitution. Pathologic efflorescences may comprise mild to serious courses of disease, and must be considered as serious risk to health in this day and age.

It is therefore the object of the present invention to provide a compound or a mixture that is suitable in minimizing or preventing skin alterations. It is also the object of the present invention to provide a compound or a mixture that protects the skin against negative influences on aesthetic and health of the skin. A compound or a mixture shall be provided for use in the prevention or treatment of impairment of the skin induced by airborne particles, of regenerative functionality of skin cells, and of health of the skin and thus of human. Furthermore, a compound or a mixture shall be provided which prevents or reduces cosmetic efflorescences and pathologic efflorescences caused by airborne particles. It is also the object of the present invention to provide a compound or a mixture for use in the prevention or treatment of the afore-mentioned skin alterations. The compound or the mixture shall exhibit an inhibitory effect on gene expression induced by airborne particles and on the impacts resulting thereof. The compound shall further inhibit or prevent health-damaging effects on the skin, caused by particles containing polycyclic aromatic hydrocarbons. Moreover, a composition containing the compound or the mixture described before for use in the prevention or treatment of cosmetic and pathologic efflorescences of human or animal skin shall be provided, and a composition for use in the prevention or treatment of skin ageing, pigmentation, hyperpigmentation, depigmentation, or hypopigmentation in human, being induced by airborne particles. Another object of the present invention is to provide cosmetic formulations and formulations for medical products comprising the compositions described before comprising the at least one compound for oral, nasal and topical administration. It is the object of the invention to provide cosmetic products and medical products for prevention or treatment of cosmetic or pathologic efflorescences caused by airborne particles.

It now has been found that compatible solutes, such as ectoine, hydroxyectoine, homoectoine and their derivatives, are suitable in prevention or treatment of cosmetic and pathologic efflorescences caused by airborne-particles, and exhibit surprising effects against alteration in pigmentation, hyperpigmentation and skin cancers (benign or malign), being induced by airborne particles.

Therefore, one subject matter of the present invention is a compatible solute or solute mixture for use in the prevention or treatment of cosmetic or pathologic efflorescences caused by airborne particles comprising at least one compound, which is selected from compounds of formula I, of formula II, physiologically compatible salts of formula I and formula II, stereoisomeric forms of the compound of formula I, formula II and physiologically compatible salts of the stereoisomeric forms, or a mixture of at least two of the afore-mentioned compounds. In formula I and in formula II mean

R1=H or alkyl,

### R2=H, COOH, COO-alkyl or CO—NH—R5,

R3 and R4 each independently H or OH,
R5=H, alkyl, an amino acid residue, dipeptide residue, or tripeptide residue
n=1, 2 or 3, and
alkyl=alkyl group having C₁-C₄ carbon atoms.

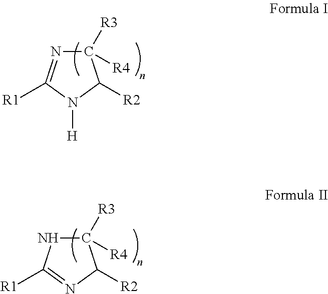

Within the meaning of the invention, alkyl comprises linear, cyclic and branched alkyl groups comprising methyl (—CH3), ethyl (—C2H5), propyl (—CH2CH2CH3 or —CH(CH3)2), and butyl (—CH2CH2CH2CH3, H3C(CH)CH2CH3, —CH2CH(CH3)2 and C(CH3)3). Linear alkyl groups are preferred, and the methyl group is particularly preferred.

Amino acid residues derive from the appropriate amino acids and their stereoisomeric forms, such as L- and D-forms, and comprise the amino acids alanine, ss-alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine, y-aminobutyrate, N-acetyllysine, N-acetylornithine, Ny-acetyldiaminobutyrate, and Na-acetyldiaminobutyrate. L-amino acids, such as L-cysteine, L-valine, L-arginine, L-asparagine, L-histidine, L-tryptophan, L-phenylalanine, and L-lysine are preferred. The amino acid residues of amino acids alanine, L-alanine, asparagine, aspartic acid, glutamine, glutamic acid, glycine, serine, threonine, valine, y-aminobutyrate, N-Acetyllysine, N-acetylornithine, Ny-acetyldiaminobutyrate, and Na-Acetyldiaminobutyrate are particularly preferred.

Dipeptide residues are composed of two amino acids and comprise linear and cyclic dipeptide residues, wherein linear dipeptide residues have one and cyclic dipeptide residues have two peptide bond(s). Tripeptide residues are composed of three amino acids and comprise linear and cyclic tripeptide residues, which have three peptide bonds in the case of linear structure or four peptide bonds in the case of cyclic structure. A peptide bond is an amide bond (—CO—NH—) between the nitrogen atom of the amine group of a first amino acid and an oxygen atom of the carboxy group of a second amino acid. Preferred dipeptide residues and tripeptide residues are composed of the afore-mentioned amino acids and, particularly preferably, of the particularly preferred amino acids described before.

Physiological salts of the compound of formula I and of the compound of formula II comprise alkali, earth alkali or ammonium salts, such as Na-, K-, Mg- or Ca-salts, as well as salts derived from organic bases, e.g. aliphatic or aromatic amines, such as triethylamine or tris-(2-hydroxyethyl)-amine. Preferred physiologically compatible salts of the compounds of formula I and of formula II are obtained by reaction with inorganic acids, such as hydrochloric acid, sulfuric acid and phosphoric acid, or with organic carboxylic acids or sulfonic acids, such as acetic acid, citric acid, benzoic acid, maleic acid, fumaric acid, tartaric acid, and p-toluene sulfonic acid.

Another subject matter of the present invention is the use of the compounds of formula I and of formula II, physiologically compatible salts of formula I and formula II, stereoisomeric forms of the compound of formula I, formula II, and physiologically compatible salts of the stereoisomeric forms, or of a mixture, as well as of preferred embodiments described herein, for the production of a cosmetic product or medical product for prevention or treatment of cosmetic or pathologic efflorescences caused by airborne particles.

In the present invention, a distinction is made between cosmetic efflorescence occurring without health-damaging impairment of human, but is pertinent to psychological well-being of human, and the physiologically pathologic efflorescence causing health-damaging impairment right up to serious and life-threatening diseases, such as skin cancer, in humans and animals. Cosmetic efflorescences comprise skin ageing, collagen degradation, pigmentation or alteration in pigmentation, and pathologic efflorescences comprise de-/hypopigmentation, hyperpigmentation, skin cancer and melanomas, in each case being induced by airborne particles.

Compounds wherein R1 is a hydrogen atom or a methyl group (CH3), R2 is a hydrogen atom or COOH, R3 and R4 are, each independently, a hydrogen atom or OH, and n is equal to 2 are preferred compounds of formula I and of formula II. In particular, preferred compounds according the afore-mentioned definitions include 1,4,5,6-tetrahydro-2-methyl-4-pyrimidine carboxylic acid (ectoine) und 1,4,5,6-tetrahydro-5-hydroxy-2-methyl-4-pyrimidine carboxylic acid (hydroxyectoine), as well as physiologically compatible salts and stereoisomeric forms of the afore-mentioned compounds. The isomeric compounds of formula I and of formula II, (S)-1,4,5,6-tetrahydro-2-methyl-4-pyrimidine carboxylic acid (S-ectoine) und (S,S)-1,4,5,6-tetrahydro-5-hydroxy-2-methyl-4-pyrimidine carboxylic acid ((S,S)-hydroxyectoine), are preferred.

Compounds of formula I and of formula II, wherein R1 is a hydrogen atom or a methyl group (CH3), R2 is a hydrogen atom or COOH, R3 and R4 are, each independently, a hydrogen atom or OH, and n is equal to 3 or 4, are more preferred for use in the prevention or treatment of cosmetic or pathologic efflorescences caused by airborne particles. In particular, preferred compounds according to the afore-mentioned definitions include (S)-4,5,6,7-tetrahydro-2-methyl-1H-[1,3]-diazepine 4-carboxylic acid (homoectoine) with n equal to 3 und 3,4,5,6,7,8-hexahydro-2-methyl-1,3-diazocine 4-carboxylic acid (HHMDCA) with n equal to 4, as well as physiologically compatible salts and stereoisomeric forms of the afore-mentioned compounds.

The compounds described before can be present as optical isomers, diastereomers, racemates, zwitterions, cations, or as a mixture of at least two of the afore-mentioned forms. Isomers comprise (R,R)-, (R,S)-, (S,S)- and (S,R)-configurations of the compounds of formula I and of formula II. Aside from isomers, diastereomers, racemates, zwitterions, cations, and mixtures of the afore-mentioned compounds are also a subject matter of the invention. Derivatisations can be performed with hydroxy acid derivatives, sulfonic acid derivatives, carboxylic acid derivatives, such as amides, esters etc., carbonyl-, ether-, alkoxy- and hydroxyl-groups. A possible derivative, without being limited thereto, is (S,S)-alpha-amino-beta-hydroxyectoine.

The terminology “solute” shall be synonymously understood as “compatible solute”, and “mixture” shall be synonymously understood as “solute mixture”. “Solute mixture” is always related to a mixture of at least two solutes of formula I and/or formula II. “Ectoine”, “hydroxyectoine” and “homoectoine” always comprise all stereoisomeric forms of the respective compound. Specific isomers are marked as such. Within the meaning of the invention, “airborne particles” means particulates from the airborne particles according to specification of ISO 4425, WHO/SDE/OEH/99.14 and DIN EN 481.

A compatible solute or a solute mixture comprising at least two of the mentioned compounds of the preceding definition is preferred, wherein the at least one compound is selected from S-ectoine, R-ectoine, (S,S)-hydroxyectoine, (S,R)-hydroxyectoine, (R,S)-hydroxyectoine, (R,R)-hydroxyectoine, and S-homoectoine, the physiologically compatible salts of S-ectoine, R-ectoine, (S,S)-hydroxyectoine, (S,R)-hydroxyectoine, (S,R)-hydroxyectoine, (R,S)-hydroxyectoine, (R,S)-hydroxyectoine, (R,R)-hydroxyectoine and S-homoectoine, the amides and esters of the afore-mentioned compounds, or is a solute mixture of at least two of the afore-mentioned compounds. S-enantiomer according to CIP priority rules corresponds to L-enantiomer according to Fisher projection, and R-enantiomer according to CIP priority rules corresponds to D-enantiomer according to Fisher projection. Particularly preferred compounds of formula I and of formula II for use in the prevention or treatment of cosmetic or pathologic efflorescences caused by airborne particles are listed below and illustrated by their structure.

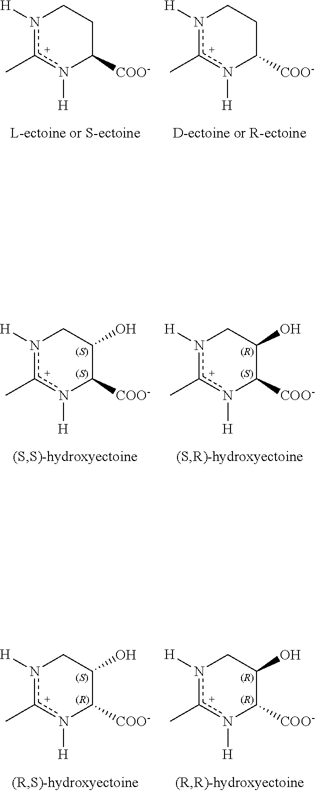

In a preferred embodiment of the solute or solute mixture according to the invention for use in the prevention or treatment of cosmetic or pathologic efflorescences caused by airborne particles, the at least one compatible solute is present in enantiopure form with a purity of greater than or equal to 90%, preferably greater than or equal to 95%, greater than or equal to 97%, greater than or equal to 99%, particularly preferably equal to 100%. This means that, relating to the solute mixture, the mixture of two compounds have the respective compound in enantiopure form, and preferably has no contamination of the selected compound by its isomer.

Enantiopure forms of the solute or solute mixture according to the invention preferably have S- and/or (S,S)-isomers.

Preferred forms of the solute according to the invention include S(L)-ectoine, R(D)-ectoine, (S,S)-hydroxyectoine, (S,R)-hydroxyectoine, (R,S)-hydroxyectoine, and (R,R)-hydroxyectoine, as well as solute mixtures of at least two of the afore-mentioned compounds.

In a preferred enantiopure solute mixture, S-ectoine and (S,S)-hydroxyectoine each are present with a purity of greater than or equal to 90%, greater than or equal to 95%, preferably greater than or equal to 97%, greater than or equal to 99%, particularly preferably equal to 100%. This solute mixture thus preferably has less than or equal to 10%, less than or equal to 5%, preferably less than or equal to 3%, less than or equal to 1%, particularly preferably equal to 0% R-ectoine, or (R,S)-/(S,R)- or (R,R)-hydroxyectoine.

In a special embodiment of the invention, the following racemates are preferred:
S-ectoine and R-ectoine
(S,S)-hydroxyectoine, (S,R)-hydroxyectoine, (R,S)-hydroxyectoine and (R,R)-hydroxyectoine, or
S-homoectoine and R-homoectoine.

Another subject matter of the present invention is a compatible solute of formula I and/or of formula II, as well as the solute mixture as described above, wherein at least one of the compounds is an expression effector, in particular an expression inhibitor, of genes associated with efflorescences. Preferably, the at least one compound of formula I or formula II of the solute or the solute mixture is an expression inhibitor of the expression of genes associated with efflorescences comprising the genes ICAM-1, POMC, MMP1 Cyp1a2, and/or Cyp1a1, which is induced by airborne particles, in particular by particulates contained therein. Within the meaning of the invention, inhibition comprises reduction of the expression induced by airborne particles, right up to complete inhibition of the expression of the afore-mentioned genes (see FIGS. 1,2 and 3, and table 3).

Consequently, another subject matter of the present invention is the use of the compatible solute or solute mixture according to the invention for the inhibition of expression of the genes POMC, Cyp1a1 and/or MMP1, which is induced by airborne particles, in the prevention or treatment of efflorescences described here (FIGS. 1, 2 and 3).

Preferably, the expression is inhibited by a factor of 1±1.00 to a factor of 20, based on the total content of 18s-rRNA. Preferably, the solute according to the invention of formula I or of formula II, as well as the solute mixture, preferably comprising S-ectoine and (S,S)-hydroxyectoine, attains an inhibitory effect by a factor of at least 5 on the expression of genes associated with efflorescences, which is induced by airborne particles.

Preferably, the solute according to the invention of formula I or of formula II, as well as the solute mixture, preferably comprising S-ectoine and (S,S)-hydroxyectoine, attains an inhibition of gene expression, which is induced by airborne particles, of greater than or equal to 20%, preferably greater than or equal to 40% to greater than or equal to 90%, preferably greater than or equal to 95% compared to expression without influence by airborne particles. Particularly preferably, the inhibitory effect described before is in a range of greater than or equal to 40% to less than or equal to 90% by weight, in particular based on Cyp1a1-expression (see FIG. 1, table 3).

Within the meaning of the invention, an expression effector is a molecule affecting the expression of a target gene. The compounds of formula I or of formula II are those molecules. Preferably, the expression effector has an inhibitory effect on the expression of the target gene. Within the meaning of the invention, the target gene is a gene, whose expression is induced and/or enhanced by the influence of airborne particles. Genes being inducible by airborne particles comprise the genes ICAM-1, POMC, MMP1, Cyp1a2 and Cyp1a1.

Particularly preferably, ectoine, S-ectoine, S-homoectoine, hydroxyectoine and/or (S,S)-hydroxyectoine, as well as the mixture of S-ectoine and (S,S)-hydroxyectoine has an inhibitory effect on the expression of the genes ICAM-1, POMC, MMP1, Cyp1a2 and Cyp1a1. The afore-mentioned compounds are preferably used as expression inhibitor for use in the prevention or treatment of cosmetic or pathologic efflorescences caused by airborne particles.

S-ectoine and (S,S)-hydroxyectoine exhibit traceably (see example 6) an inhibitory effect on the expression of Cyp1a1 and thus an inhibition of cancer-causing metabolisation of polycyclic aromatic hydrocarbons, such as SRM1650 or SRM2975 (see FIG. 1, table 3).

The protective function against skin cancer of the solute according to the invention of formula I and of formula II in the use for prevention or treatment of pathologic efflorescences is thereby proved. In this way, the solute according to the invention, preferably S-ectoine and/or (S,S)-ectoine, protects human skin, in particular keratinocytes and melanocytes, against pathologic efflorescences.

Inhibition of expression of the POMC gene proves reduced production of melanotropins (MSH), such as alpha-MSH, beta-MSH and gamma-MSH, which are responsible for melanin synthesis as well as melanocyte expansion in pigment-forming melanocytes. Consequently, inhibition of POMC expression results in reduced pigmentation in the skin (example 4-6, FIG. 2, table 3). This proves the effectiveness of the used solutes within the meaning of the invention in the prevention or treatment of cosmetic efflorescences, such as, for example, pigmentation, pigmented spots, ageing spots, or senile atrophy, as well as pathologic efflorescences, such as hyperpigmentation.

The same applies to atrophies in general, in particular to atrophies occurring as side effect by high and prolonged intake of medication, such as corticoid steroid applications on the skin. Within the meaning of the invention, those atrophies are summarised under acquired melanotic and non-melanotic hyperpigmentation of the skin, and occur, in particular, as side effect by intake of medication comprising antimycotic, anaesthetic, hormone preparations (e.g. steroids, testosterone), antibiotic (e.g. meta-/Tetra-/mino-/tigecycline), antivirals, (e.g. zidovudine), antineoplastics, immunosuppressant (e.g. cyclosporine A and quinacrine), hyaluronic acid, psycholeptics, and antidepressants.

The Inhibition of the expression of the Cyp1a1 gene indicates downregulation of the detoxification reaction of the skin. Consequently, when inhibiting the expression of Cyp1a1 gene fewer environmental toxins, in particular comprising polycyclic aromatic hydrocarbons, are metabolised in cancer-causing intermediates (example 4-6, FIG. 1, table 3). This proves the effectiveness of the used solutes according to the invention in the prevention or treatment of pathologic efflorescences, such as benign tumors, melanomas, carcinomas, spinocellular carcinomas and others.

The Inhibition of the expression of the MMP1 gene proves downregulation of the collagenase. This proves that less collagen is degraded, hence in total less skin tissues are degraded, and the skin thus ages more slowly. This proves the effectiveness of the used solutes within the meaning of the invention in the prevention or treatment of cosmetic efflorescences, such as senescence of the skin, premature senescence, or elasticity displacement, in particular of those accompanying with changes in the biological activity of keratinocytes. Consequently, the compatible solute according to the invention, preferably ectoine and/or hydroxyectoine, can be used for prevention or treatment of benign or malign cancers, also referred to as tumor formation (FIG. 3, table 3).

Expression of ICAM-1 is a proof of inflammatory reactions in the cells. The solute according to invention according to formula I and/or formula II, preferably ectoine and/or hydroxyectoine, is suitable as inflammation inhibitor of inflammatory reactions of the skin, which are induced by airborne particles.

In a preferred embodiment of the invention, the compatible solute or the solute mixture described before is used in the prevention or treatment of cosmetic or pathologic efflorescences caused by airborne particles, which is an efflorescence of melanocytes and/or keratinocytes. In particular, it is a biochemically and/or molecular biologically detectable alteration of melanocytes and/or keratinocytes.

In particular, the efflorescence is caused by airborne particles interacting with receptors of the outer plasma membrane surface, of the cytoplasmic (inner) membrane surface and/or of the membrane surface of organelles in the cell interior. The interaction of airborne particles and the afore-mentioned receptors affects signaling pathways associated therewith, in particular membrane-bound signaling pathways. The influence of airborne particles results, intracellularly, in a direct influence on protein biosynthesis of transmembrane proteins and cytosolic proteins. In doing so, airborne particles cause, via feedback, shifts in equilibrium of signaling cascades and of gene expression, preferably of the genes ICAM-1, POMC, MMP1, Cyp1a2 und/oder Cyp1a1.

Preferably, the signaling pathways described before are such regulated by the solute or solute mixture according to the invention that they counteract the effects of the airborne particles, resulting in inhibition of expression of the genes ICAM-1, POMC, MMP1, Cyp1a2 and/or Cyp1a1. Particularly preferably, the solutes according to the invention, preferably S-ectoine and/or (S,S)-hydroxyectoine, interact with receptors of the outer plasma membrane surface or the cytoplasmic (inner) membrane surface, effecting inhibition of expression of the genes ICAM-1, POMC, MMP1, Cyp1a2 und/oder Cyp1a1. In this way, preferably S-ectoine and/or (S,S)-hydroxyectoine protect the signaling cascades of the skin cells, described before, against influence by airborne particles, in particular nanoparticles.

In a special embodiment, the solutes according to the invention, preferably S-ectoine and/or (S,S)-hydroxyectoine, penetrates into the ion channels of membrane, or via membrane-bound translocation systems into the cytosol, therein affecting signaling cascades, which preferably result in inhibition right up to complete inhibition of at least one of the genes ICAM-1, POMC, MMP1, Cyp1a2 and/or Cyp1a1.

In another embodiment, the at least one compatible solute or the solute mixture, according to the definition described before of formula I and of formula II is used for prevention or treatment of cosmetic or pathologic efflorescences caused by airborne particles, wherein the efflorescence comprises an alteration of at least one cell layer, in particular of at least one cell type, in at least one part of the skin, being phenotypically visible on the skin surface. Preferably, it is an alteration of keratinocytes and/or melanocytes, being micro-/macroscopically visible on the skin surface.

The efflorescence being visible on the skin surface, can be visible based on alteration in pigmentation, such as pigment spots, depigmentation, or hypopigmentation, and hyperpigmentation. Furthermore, the visible efflorescence can comprise benign tumor formation or malign tumor formation with or without accompanying alteration in pigmentation. Preferably, the visible efflorescence is the phenotype of induction or enhancement of gene expression of ICAM-1, POMC, MMP1, Cyp1a2 and/or Cyp1a1, which have occurred by airborne particles on biochemical and/or molecular biological level. Inhibition of at least one of the genes ICAM-1, POMC, MMP1, Cyp1a2 and/or Cyp1a1 is obtained by the solute according to the invention of formula I and/or formula II, as well as the solute mixture, preferably comprising S-ectoine and/or (S,S)-hydroxyectoine.

In this way, the solute according to the invention of formula I and/or of formula II, as well as the solute mixture, preferably comprising S-ectoine and/or (S,S)-hydroxyectoine, is used for prevention or treatment of cosmetic efflorescences or pathologic efflorescences, wherein the effectiveness is micro-/macroscopically visible in histology and is biochemically and/or molecular biologically detectable on cell level (see examples).

The solute or solute mixture within the meaning of the invention, being suitable for prevention or treatment, can be used in case of efflorescences of different types of tissue. In particular, mucus-free tissues are comprised. Mucus-free tissues comprise all tissues, such as muscle tissue, nerve tissue and connective tissue, wherein mucous membranes are preferably exempted (eye, nose, lips, mouth, bronchial tubes, lung, stomach, intestine, anus, vagina, vulva, penis).

The efflorescence can be an alteration of cutis, preferably of epidermis comprising at least one alteration of a tissue selected form stratum corneum, stratum lucidum, stratum granulosum, stratum spinosum, stratum basale and/or stratum germinativum (=stratum spinosum+stratum basale), dermis comprising stratum paillare and stratum reticulare, and/or of subcutis comprising connective tissue with fibroblasts, endothelial cells, collagen and fat cells.

Another subject matter of the present invention is a solute mixture for use in the prevention or treatment of cosmetic or pathologic efflorescences caused by airborne particles, wherein the solute mixture comprises at least two compounds of formula I and/or of formula II.

The solute mixture according to the invention, described before, for use in the prevention or treatment of cosmetic or pathologic efflorescences caused by airborne particles comprises, based on the sum of all compounds in the solute mixture having a total content of 100% by weight,
an amount of S-ectoine in a range of greater than or equal to 50% by weight to less than or equal to 100% by weight, and an amount of (S,S)-hydroxyectoine in a range of greater than or equal to 50% by weight to less than or equal to 100% by weight, and preferably,
an amount of S-ectoine in a range of greater than or equal to 75% by weight, preferably greater than or equal to 85% by weight to less than or equal to 95% by weight, and an amount of (S,S)-hydroxyectoine in a range of greater than or equal to 5% by weight to less than or equal to 25% by weight, preferably less than or equal to 15% by weight.

In a special embodiment of the solute mixture according to the invention is
the amount of S-ectoine less than or equal to 70% by weight, preferably less than or equal to 65% by weight, less than or equal to 60% by weight, less than or equal to 55% by weight, particularly preferably greater than or equal to 50% by weight, and
the amount of (S,S)-hydroxyectoine greater than or equal to 30% by weight, preferably greater than or equal to 35% by weight, greater than or equal to 40% by weight, greater than or equal to 45% by weight, particularly preferably less than or equal to 50% by weight.

In a special embodiment, the solute mixture comprises a mixture of two compounds of formula I and/or of formula II each having a content of greater than or equal to 60% by weight of the first compound to less than or equal to 40% by weight of the second compound. Preferably, said solute mixture comprises greater than or equal to 60% by weight, particularly preferably greater than or equal to 70% by weight S-ectoine and less than or equal to 40% by weight, particularly preferably less than or equal to 30% by weight (S,S)-hydroxyectoine.

In a special embodiment, the solute mixture comprises a mixture of two compounds of formula I and/or of formula II having a content of 50% by weight S-ectoine and 50% by weight (S,S)-hydroxyectoine.

Another subject matter of the present invention is the use of the compatible solute or solute mixture according to the invention, as described above, for prevention or treatment of cosmetic or pathologic efflorescences, caused by airborne particles which comprise particulate and/or fibrous environmental noxae according to specification of DIN EN 481, WHO/SDE/OEH/99.14 or ISO 4225.

In a special embodiment, the cosmetic and/or pathologic efflorescences are caused by nanoparticles, in particular carbon nanoparticles, particles containing polycyclic aromatic hydrocarbons, and/or diesel soot particles, in particular comprising at least one compound selected from 2-nitrofluorene, 6-nitrobenz[a]pyrene, 7-nitrobenz[a]anthracene, 9-fluorenone, benzo[k]fluoranthene, benzo[e]pyrene, chrysene, indeno[1,2,3,-cd]pyrene, perylene, phenanthrene, pure carbon (greater than 90% to 100%), and hydrocarbons. Carbon particles comprise, for example, Printex90, as well as Huber 990, and diesel-soot particles comprise SRM1650, as well as SRM2975, as described in example 1.

Cosmetic or pathologic efflorescences caused by airborne particles comprise, in particular, ultra-fine and/or nano-fine particles, whose mobility equivalent diameter is less than or equal to 100 nm, and/or fibrous particles having a length of greater than or equal to 5 μm and a diameter of less than or equal to 3 μm.

Elongated dust particles, in particular fibrous particles of the definition described before have (with high bio-persistency) cancer-causing effect, in particular particles according to WHO/SDE/OEH/99.14, for example asbestos and AMF (=artificial mineral fibres).

In particular, the airborne particles comprise particles having a particle size of less than or equal to 100 μm, in particular less than or equal to 50 μm, less than or equal to 20 μm (inhalable particles), less than 15 μm, less than or equal to 10 μm (thoracic particles), and less than or equal to 7 μm (tracheobronchialic particles or dust), less than or equal to 3 μm (alveolar particles), preferably less than or equal to 1 μm (fine dust particles), and particularly preferably, less than or equal to 0.1 μm (ultra-fine dust/nanoparticles).

Within the meaning of the invention airborne particles preferably meet the specifications of norms DIN EN 481, WHO/SDE/OEH/99.14, or ISO 4225).

DIN EN 481 describes the “Determinations of conventions of particle size fractions for measurement of airborne particles at the workplace” and distinguishes inhalable dust fraction, alveolar fraction, thoracic, extrathoracic and tracheobronchialic fraction, as well as further kinds of dust, such as fibres and ultra-fine dust.

DIN EN 481 specifies
the inhalable dust fraction (previously referred to as total dust):
   comprises the mass fraction of all suspended matter in the air being inhaled via mouth and nose, and
   all particles less than or equal to 100 μm,
the alveolar fraction (fine dust):
   the mass fraction of the inhaled particles, which penetrates into not-ciliated respiratory tract
   particles less than or equal to 10 μm.

ISO 4225 specifies particles as being small, separated particles of a solid or liquid substance, and nanoparticles as being particles having orientation in nanoscale range in all three spatial dimensions. The person skilled in the art refers further definition from ISO 4225. Preferably, the present invention is related to cosmetic and pathologic efflorescences caused by nanoparticles.

In WHO/SDE/OEH/99.14, reference is also made to ISO 4225. According to WHO/SDE/OEH/99.14, dust comprises small particles being less than or equal to 75 μm in diameter, and airborne particles usually have a diameter in the range of 1 μm to 100 μm. Further definitions may be gathered from WHO/SDE/OEH/99.14.

In a special embodiment of the present invention, the at least one solute of formula I and/or of formula II, or the solute mixture comprising at least two solutes of formula I and/or of formula II is biobased and therefore of biological origin. The biobased solute or solute mixture is preferably used in the prevention or treatment of cosmetic and/or pathologic efflorescences caused by airborne particles.

Within the meaning of the invention, biobased or of biological origin means that the compound of formula I and/or of formula II is produced by or in an organism. Preferably, the organism is a microorganism and, particularly preferably, the microorganism is a halophilic bacterium comprising Ectothiorhodospira halochloris, Halomonas elongata, Marinococcus halophilus, Brevibacterium linens, Halomonas SPC1, Volcaniella eurihalina, Deleya salina, Bacillus pantothenticus, Bacillus halophilus, Vibrio costicola and Streptomyces parvulust. Within the meaning of the invention, the biobased solute of formula I and/or of formula II, or the solute mixture described above, preferably S-/R-ectoine and/or (S,S)-/(S,R)-/(R,S)-/(R,R)-hydroxyectoine, is produced by Halomonas elongata, Brevibacterium lines or Marinococcus halophilus and is obtained from said bacteria.

Compounds of formula I or of formula II, as well as the solute mixture comprising S-ectoine and (S,S)-/(S,R)-/(R,S)-/(R,R)-hydroxyectoine, being produced in biotechnologically modified organisms, preferably in recombinant microorganism comprising, but not limited to the afore-mentioned strains, are also biobased or of biological origin since the afore-mentioned compounds are produced by equipment of biological cell rather than synthetically in chemistry laboratory without involvement of a biological organism.

Compounds of formula I or of formula II, having identical structure, being synthetically produced outside of an organism described before, are not included by the definition of biobased solutes.

Another subject matter of the present invention is a method for the production of the at least one compound according to the invention of formula I or of formula II, of the solute or solute mixture, the use of the afore-mentioned compounds in a method for the production of the solute or solute mixture according to the invention, as well as use in a method for the production of a composition comprising the at least one solute or solute mixture according to the invention, preferably S-ectoine and/or (S,S)-hydroxyectoine. Preferably, the method according to the invention for the production of the solute or solute mixture, as well as of the composition according to the invention meets the requirements of ISO 13485.

Another subject matter of the present invention is a composition comprising at least one compatible solute or one solute mixture comprising at least two compatible solutes for use in the prevention or treatment of cosmetic or pathologic efflorescences caused by airborne particles, wherein the compatible solute or the solute mixture comprises at least one compound, which is selected from compounds of formula I, of formula II, physiologically compatible salts of formula I and formula II, stereoisomeric forms of the compound of formula I, formula II, and physiologically compatible salts of the stereoisomeric forms, or a mixture of at least two of the afore-mentioned compounds.

The definition made above of formula I and of formula II, as well as the definition of the residues and preferred compounds apply accordingly.

Preferred compositions comprise the at least one compatible solute or the solute mixture in the composition in an amount of greater than or equal to 0.0001% by weight to less than or equal to 50% by weight, based on the total content of the composition. A range of greater than or equal to 0.001% by weight, greater than or equal to 0.01% by weight, greater than or equal to 0.1% by weight, particularly preferably greater than or equal to 1.0% by weight to less than or equal to 40% by weight, less than or equal to 30% by weight, less than or equal to 20% by weight, particularly preferably less than or equal to 10% by weight, is particularly preferred.

In a particularly preferred composition, the at least one compatible solute or the solute mixture is present in the composition in an amount of greater than or equal to 0.0001% by weight to less than or equal to 10% by weight, based on the total content of the composition, preferably greater than or equal to 0.001% by weight to less than or equal to 8% by weight, preferably to less than or equal to 6% by weight, particularly preferably less than or equal to 5% by weight.

Another subject matter of the present invention is the composition described before, wherein it is used in the prevention or treatment of cosmetic or pathologic efflorescences, caused by particulate and/or fibrous environmental noxae according to specification of DIN EN 481, WHO/SDE/OEH/99.14 or ISO 4225. The particles being affected by the norms have already been described above, and reference is made here to the corresponding description texts.

Preferably, the composition according to the invention comprises at least one compound of formula I and/or of formula II selected from S-ectoine, R-ectoine, (S,S)-hydroxyectoine, (S,R)-hydroxyectoine, (R,S)-hydroxyectoine, (R,R)-hydroxyectoine and S-homoectoine, the physiologically compatible salts of S-ectoine, R-ectoine, (S,S)-hydroxyectoine, (S,R)-hydroxyectoine, (R,S)-hydroxyectoine, (R,R)-hydroxyectoine, and S-homoectoine, the amides and esters of the afore-mentioned compounds, or a solute mixture of at least two of the afore-mentioned compounds.

Particularly preferably, at least one of the afore-mentioned compatible solutes or the solute mixture is present in the composition with an amount of greater than or equal to 0.0001% by weight to less than or equal to 10% by weight, based on the total content of the composition.

In a special embodiment of the present invention, the composition according to the invention, preferably comprising S-ectoine and/or (S,S)-hydroxyectoine, for prevention or treatment of cosmetic or pathologic efflorescences caused by airborne particles is present in solid or liquid form, selected from
i) solid forms comprising powder, tablets, granule, form-coated tablet, dragee, capsules, effervescent tablets, powder, and soap,
ii) liquid forms comprising solution, injection, infusion, tincture, infusion solution, suspension, syrup, juice, emulsion, application, foam, cream, lotion, surfactant-containing cleaning preparation, oil, and/or
iii) mixtures comprising spray, aerosols, inhalant, ointment, paste.

Formulations for topical administration on the skin surface comprising at least one solute of formula I or of formula II, preferably S-ectoine and/or (S,S)-hydroxyectoine, particularly preferably being in the composition in an amount of greater than or equal to 0.0001% by weight to less than or equal to 10% by weight, based on the total content of the composition are preferred forms for prevention or treatment of pathologic efflorescences, such as benign and malign skin cancer in general, hypopigmentation, hyperpigmentation, or one of the diseases listed below.

Cosmetic formulations comprising mixtures selected from lipstick, lip balm, mascara, eyeliner, eyeshadow, blusher, powder, emulsion make-up or wax make-up, are used for prevention or treatment of cosmetic efflorescences and are also a subject matter of the present invention.

Cosmetic formulations for topical administration, for example by spraying, massaging in or applying, of the composition according to the invention particularly preferably include
i) solid forms comprising powder, powder, soap, capsules
ii) liquid forms comprising solution, injection, infusion, tincture, infusion solution, suspension, emulsion, application, foam, cream, lotion, surfactant-containing cleaning preparation, oil, and/or
iii) mixtures comprising spay, ointment or paste,
   which preferably comprise at least one solute of formula I or of formula II, preferably S-ectoine and/or (S,S)-hydroxyectoine, particularly preferably being in the composition in an amount of greater than or equal to 0.0001% by weight to less than or equal to 10% by weight, based on the total content of the composition.

For oral administration for ingestion via gastrointestinal tract by swallowing of the composition according to the invention are particularly preferred
i) solid forms comprising powder, in particular after mixing with a beverage or water, capsules, tablets, granule, film-coated tablet, dragee, and effervescent tablets
ii) liquid forms comprising solution, infusion solution, syrup, juice, and oil.

For oral administration for ingestion via blood circulation by inhaling the composition according to the invention are particularly preferred i) solid forms, such as powder, or iii) mixtures comprising spay, aerosols and inhalant.

Formulations according to the invention for prevention or treatment of cosmetic efflorescences caused by airborne particles comprise cosmetic formulations preferably meeting the requirements of regulation (EG) No. 1223/2009 and the requirements of regulation (EG) No. 358/2014, and particularly preferably comprise at least one solute of formula I or of formula II, preferably S-ectoine and/or (S,S)-hydroxyectoine, being in the composition in an amount of greater than or equal to 0.0001% by weight to less than or equal to 10% by weight, based on the total content of the composition.

Each composition according to the invention can comprise known excipients from the state of the art in the selected formulation.

Excipients comprise carriers, preservatives, antioxidants, stabilizers, solubilizers, vitamins, colorants, smell improver.

Carriers, in particular for cosmetic formulations, comprise animal and vegetable fats, waxes, paraffins, starch, tragacanth, cellulose derivatives, polyethylene glycols, silicons, bentonites, silicic acid, talcum, and zinc oxide, or mixtures of at least two of the afore-mentioned substances. The afore-mentioned carriers are particularly well-suited for ointments, pastes, creams and applications comprising at least one solute of formula I or of formula II, preferably S-ectoine and/or (S,S)-hydroxyectoine.

Carriers for powders or sprays comprising at least one solute of formula I or of formula II, preferably S-ectoine and/or (S,S)-hydroxyectoine comprise lactose, talcum, silicic acid, aluminium hydroxide, calcium silicate and polyamide powder, or mixtures of at least two of the afore-mentioned substances. Sprays can additionally comprise common propellants, for example hydrochlorfluorocarbons, propane/butane or dimethylether.

Solutions and emulsions comprising at least one solute of formula I or of formula II, preferably S-ectoine and/or (S,S)-hydroxyectoine, can comprise usual carriers, such as solvents, solubilizers and emulsifiers, for example water, ethanol isopropanol, ethylcarbonate, ethyl-acetate, benzylalcohol, benzylbenzoate, propylene glycol, 1,3-butylglycol, oils, in particular cottonseed oil, peanut oil, maize germ oil, olive oil, castor oil and sesame oil, glycerin fatty acid ester, polyethylene glycol and fatty acid esters of sorbitan, or mixtures of at least two of the afore-mentioned substances.

Suspensions comprising at least one solute of formula I or of formula II, preferably S-ectoine and/or (S,S)-hydroxyectoine, comprise usual carriers, such as liquid diluents, for example water, ethanol or propylene glycol, suspension agents, for example ethoxylated isosterylalcohols, polyoxymethylene sorbitol ester and polyoxymethylene sorbitan ester, microcrystalline cellulose, aluminium metahydroxide, bentonite, agar agar and tragacanth, or mixtures of at least two of the afore-mentioned substances.

Soaps comprising at least one solute of formula I and or of formula II, preferably S-ectoine and/or (S,S)-hydroxyectoine, which can comprise carriers, such as alkali salts of fatty acids, salts of fatty acid semi-esters, fatty acid protein hydrolysate, isothionates, lanolin, fatty alcohol, vegetable oils, botanical extracts, glycerin, sugar, or mixtures of at least two of the afore-mentioned substances, are particularly well-suited for daily use.

Further excipients being usual and suitable for cosmetic formulations are known to the person skilled in the art.

In a special embodiment of the present invention, the composition according to the invention is used for prevention or treatment of cosmetic efflorescences caused by airborne particles, wherein the efflorescence is a senescence of the skin, the skin being exposed to airborne particles, exhibits each a deviating pigmentation, deviating expression of at least one gene, in particular POMC, Cyp1a1 and/or MMP1, and/or exhibits a deviating production of at least one biomolecule, in particular of those biomolecules participating in skin ageing and pigmentation of the skin, such as melanin, collagenase, P450-cytochrome enzyme, compared to the skin not being exposed to airborne particles. Biomolecules comprise DNA, RNA, proteins, enzymes, prehormones, hormones, signaling molecules, such as signaling peptides and transcription factors.

In particular, the skin being exposed to airborne particles has an increased, unnatural and uneven pigmentation. Preferably, a pigmentation being micro- or macroscopically visible, in histology. Particularly preferably, the afore-mentioned pigmentations have been caused by gene expression of ICAM-1, POMC, MMP1, Cyp1a2 and/or Cyp1a1, induced or enhanced by airborne particles (table 3).

Senescence of the skin comprise premature senescences caused by stress factors, comprising elasticity displacement, benign and malign tumors, alteration in pigmentation, or reduced adhesion of corneocytes. In particular, benign tumors and elasticity displacements can be traced to enhanced expression of MMP1-gene, and alteration in pigmentation to enhanced POMC-expression. Within the meaning of the invention, the influence of airborne particles on the skin shall be understood as being a stress factor affecting the skin.

Alteration in pigmentation comprise tanning of “too” light skin, ageing spots, yellowish-brownish appearances, which are obviously not attributed to natural tanning through sun exposure.

In a special embodiment of the invention, a composition comprising at least one solute of formula I or of formula II, or a solute mixture of at least two of the afore-mentioned solutes, particularly preferably S-ectoine and/or (S,S)-hydroxyectoine, being in the composition in an amount of greater than or equal to 0.0001% by weight to less than or equal to 10% by weight, based on the total content of the composition, is used in the prevention or treatment of cosmetic efflorescences in the form of skin ageing and/or pigmentation, in particular senile atrophy, caused by airborne particles.

Senile atrophy is a yellowish or grey-yellowish, but also brindled hyperpigmented by chronic actinic influence, loose, often dry, withered, parchment-like skin, often being infiltrated with telangiectasias (primarily in face and on thorax), as well as with clear prominence of larger venous and arterial vessels lying under the skin. The skin can exhibit increased wrinkle formation and wrinkling. Division of the skin in rough rhombic areas. Often leucomelanodermal alteration in pigmentation, reduced activity of sebaceous and sweat glands with obvious exsiccosis of the skin.

Another subject matter of the present invention is the use of the composition according to the invention comprising at least one solute of formula I or of formula II or a solute mixture of at least two of the afore-mentioned solutes, preferably S-ectoine and/or (S,S)-hydroxyectoine, in the prevention or treatment of reduced regenerative functionality of keratinocytes and/or melanocytes, in particular of at least one cell of the spinous layer (stratum spinosum), mastocyte layer (Langerhans cells) and/or basal layer (stratum basale). Regenerative functionality comprises all biological processes in stratum germinativum, participating in regeneration of the skin. In particular, biological processes regulated by the genes ICAM-1, POMC, MMP1, Cyp1a2 and/or Cyp1a1.

Another subject matter of the present invention is a composition comprising at least one solute of formula I or of formula II or a solute mixture of at least two of the afore-mentioned solutes, preferably S-ectoine and/or (S,S)-hydroxyectoine, for use in the prevention or treatment of pathologic efflorescences caused by airborne particles comprising depigmentation, hypopigmentation, hyperpigmentation, acquired melanotic and non-melanotic hyperpigmentation, incontinentia pigmenti, dermatoses, dermatoses caused by building materials or cement, pigmented spots, anuli pigmentosi, prurigo pigmentosa, peribuccal pigmentation, carcinomas, spinocellular coarcinoma, pigmented spindle-cell tumor, benign or malign tumor formation, Leschke syndrome, skin metastases, Becker's nevus, malignant melanoma, pigmented neurofibroma, urticarial pigmentosa, Peutz-Jeghers syndrome, epidermodysplasia verruciform is, tacrolimus, pimecrolimus, vitiligo, atrophy-associated diseases comprising exsiccation eczemas, elastoses, purpura senilis, lentigo solaris, or Favre-Racouchot syndrome.

Pathologic efflorescences are also summarized as such skin alterations that originally occur as cosmetic efflorescence and evolve into pathologic efflorescences in the course of disease, in particular through further influence of airborne particles or changes in the constitution of the skin cells. These efflorescences include inter alia telangiectasias, starting innocuously and evolving into collagenoses, Osler's disease/hereditary haemorrhagic telangiectasia (HHT) and mycosis fungoides.

The composition according the invention, preferably comprising S-ectoine and/or (S,S)-hydroxyectoine, is used in solid or liquid form, in particular as medical product, for prevention or treatment of pathologic efflorescences caused by airborne particles, wherein the formulation is selected from
i) solid forms comprising powder, tablets, granule, film-coated tablet, dragee, capsules, effervescent tablets, powder, and soap,
ii) liquid forms comprising solution, injection, infusion, tincture, infusion solution, suspension, syrup, juice, emulsion, application, foam, cream, lotion, surfactant-containing cleaning preparation, oil, and/or
iii) mixtures comprising spray, aerosols, inhalant, ointment, paste.

Preferred formulations according to the invention, in particular medical products, preferably comprising S-ectoine and/or (S,S)-hydroxyectoine, for prevention or treatment of pathologic efflorescences caused by airborne particles, are suitable formulations for topical administration on the skin surface. They particularly preferably comprise S-ectoine and/or (S,S)-hydroxyectoine, being in the composition in an amount of greater than or equal to 0.0001% by weight to less than or equal to 10% by weight, based on the total content of the composition.

Within the meaning of the invention, medical products include the formulations described herein comprising at least one solute of formula I or of formula II or the solute mixture of at least two of the afore-mentioned compounds, preferably S-ectoine and/or (S,S)-hydroxyectoine, for use in the prevention or treatment of human in medical therapeutic purposes. Preferably, the medical products according to the invention meet the specification and requirements of regulation 93/42/EWG.

Preferred excipients for compositions of medical products in solid or liquid form for prevention or treatment of pathologic efflorescences comprise lactose, sucrose, dextrose, mannitol, sorbitol, starch, gelatine, tragacanth, pectin, cellulose, methylcellulose, hydroxypropylmethylcellulose (HPMC), carboxymethylcellulose sodium, ethylcellulose, hydroxypropylmethylcellulose phthalate, cellulose acetate phthalate, polyvinylpyrrolidone, polyvinylalcohole, polyacrylic acid, polyethylene glycol, polyethylen oxide, sodiumdodecylsulfate, sodiumacetylstearylsulfate, and sodiumdioctylsulfosuccinate (also K salts, Ca salts).

Preferred excipients for solutions and suspensions according to the invention comprise dextrose, mannitol, tragacanth, pectin, methylcellulose, hydroxypropylmethylcellulose (HPMC), carboxymethylcellulose sodium, polyvinylpyrrolidone, polyvinylalcohole, polyacrylic acid, polyethylene glycol, polyethylene oxide, sodiumdodecylsulfate, sodiumacetylstearylsulfate, sodiumdioctylsulfosuccinate (also K salts, Ca salts), and, in particular for suspensions, cellulose.

Within the meaning of the invention, semisolid forms or mixtures preferably comprise hydroxypropylmethylcellulose (HPMC), carboxymethylcellulose sodium, polyethylene glycol, polyethylene oxide, sodiumdodecylsulfate, sodiumacetylstearylsulfate and pectin.

A preferred composition of a medical product comprising the solute according to the invention of formula I or of formula II or the solute mixture of at least two of the afore-mentioned solutes, preferably S-ectoine and/or (S,S)-hydroxyectoine, for prevention or treatment of pathologic efflorescences caused by airborne particles, comprises formulations for applying, depositing, massaging in, spraying on and/or laying on the affected skin surface.

The examples below show the effectiveness of ectoine to protect the skin against expression of the genes POMC, Cyp1a1 and MMP1, which is induced by fine and ultra-fine carbon particles as well as by diesel soot particles. In particular, ectoine prevents enhanced expression of MMP1 gene which is induced by fine carbon and diesel soot particles.

The experiments below prove the effectiveness of ectoine and related compounds of formula I and of formula II, and are suitable for use in a composition and in one of the formulations described before for prevention or treatment of pigmentation or skin ageing, caused by airborne particles.

### DESCRIPTION OF THE FIGURES

FIG. 1 shows the expression of Cyp1a1 gene relative to the total content of 18s-rRNA of keratinocytes. Keratinocytes incubated with Printex90, Huber 990, SRM1650 and SRM2975 exhibit significantly increased Cyp1a1 expression compared with untreated Keratinocytes (=blank). Cyp1a1 expression could be inhibited by pretreatment with 2 mM ectoine (model particles/ectoine). (p<0.05 and comparison to blank or p<0.05 as treated).

FIG. 2 shows the expression of POMC gene relative to the total content of 18s-rRNA of keratinocytes. Keratinocytes incubated with Printex90, Huber 990, SRM1650 and SRM2975 exhibit significantly increased POMC expression compared with untreated Keratinocytes (=blank). Cyp1a1 expression could be inhibited by pretreatment with 2 mM ectoine (model particles/ectoine). (p<0.05 and comparison to blank or p<0.05 as treated).

FIG. 3 shows the expression of MMP1 gene relative to the total content of 18s-rRNA of keratinocytes. Keratinocytes incubated with Printex90, Huber 990, SRM1650 and SRM2975 exhibit significantly increased MMP1 expression compared with untreated Keratinocytes (=blank). Cyp1a1 expression could be inhibited by pretreatment with 2 mM ectoine (model particles/ectoine). (p<0.05 and comparison to blank or p<0.05 as treated).

### EXAMPLES

### Example 1: Model Particles

TABLE 1
Used model particles:

| Model particles | Name | Source |
|---|---|---|
| ultra-fine soot particles | Printex90 | Evonik Industries, Essen, Germany |
| fine soot particles | Huber 990 | Evonik Industries, Essen, Germany |
| diesel exhaust particles | SRM1650 | National Institute of Standards and |
| | | Technology, Gaithersburg, MD, USA |
| diesel exhaust particles | SRM2975 | National Institute of Standards and |
| | | Technology, Gaithersburg, MD, USA |

The toxicity of soot particles, such as Printex90 and Huber990, has been extensively studied, and summarized by the European Commission of consumer protection in a meeting on 12 Dec. 2013. The revised report has been published as SCCS/1515/13 on 27 Mar. 2014 (ISBN 978-92-79-30120-9). Reference is made to this report and its content is incorporated by reference into the disclosure of the present invention.

### Printex90 Particle (CAS Number 1333-86-4) has the Following Composition:

Printex90 particle are ultra-fine amorphous carbon soot particles insoluble in water (>99% pure soot, PAH=0.039 ppm) having a molecular weight of 12, a density at 20° C. of 1.7 to 1.9 g/cm, melting point 3000° C., pH according to ASTM 1512 in a range of 4 to 11 (at 50 g/L water, 20° C.). The particles have a bulk density of 20-640 kg/m³. Particles having a pH value of greater than 7 correspond to furnace soot rubber.

The particle size is 7.7 nm to 28.2 nm, and has a mean particle diameter of 14.3±0.6 nm (surface 250 to 337 m²/g).

After exposition of Printex90 particles acute toxicity in rats by oral intake has been shown (LD50 at least 8000 mg/kg).

### Huber990 Particles:

Huber 990 particles are fine carbon soot particles insoluble in water, having comparable constitution to Printex90. Huber990 particles have a mean particle diameter of 260.2 nm (surface 7.9 m²/g).

SRM1650 Particles have the Following Composition:

SRM particles are complex diesel soot particles which, after a total of 200 operating hours, are obtained from heat exchangers of four diesel engines, operating and being loaded to a different degree. The obtained particles correspond to diesel soot particles occurring in environment, and have the composition described below. In salmonella mutagenesis assay (Maron, D., and Ames, B., “Revised Methods for Salmonella Mutagenicity Test”, Mutation Research, 113: 1723-212) mutagenic activity of 17.5±1.5% has been shown for SRM1650 particles, based on the average methylene chloride content which was isolated from SRM1650 particles.

### Composition According to Available Certificate (See Source, Table 1)

| Component | Concentration [μg] | Standard deviation (±) |
|---|---|---|
| 1-Nitropyrene | 19 | 2 |
| Benz[a]anthracene | 6.5 | 1.1 |
| Benzo[a]pyrene | 1.2 | 0.3 |
| Benzo[ghi]perylene | 2.4 | 0.6 |
| Fluoranthene | 51 | 4 |
| Pyrene | 48 | 4 |

### Non-Certified Composition Available at Afore-Mentioned Source (Table 1):

| Component | Concentration [μg] |
|---|---|
| 2-Nitrofluorene | 0.2 |
| 6-Nitrobenzo[a]pyrene | 1.6 |
| 7-Nitrobenz[a]anthracene | 2.8 |
| 9-Fluorenone | 33 |
| Benzo[k]fluoranthene | 2.1 |
| Benzo[e]pyrene | 9.6 |
| Chrysene | 22 |
| Indeno[1,2,3-cd]pyrene | 2.3 |
| Perylene | 0.13 |
| Phenanthrene | 71 |

### SRM2975 Particles:

SRM2975 particles have comparable constitution to SRM1650 particles, but have another particle size. SRM2975 particles have a particle size of 10 μm to 50 μm.

The model particles were suspended in phosphate buffered saline solution and treated with ultrasound for 1 minute (Printex90, SRM1650 and SRM2975) or 90 seconds (Huber990). Subsequently, addition to keratinocytes was done immediately.

The concentration of the respective model particles used for treatment of keratinocytes was 1.5 μg cm⁻², respectively, based on the total area of the keratinocytes surface in subconfluent cell layer.

The incubation time of keratinocytes under the influence of model particles was 24 hours.

### Example 2: Cell Cultures

Human epidermal keratinocytes were taken from healthy patients during abdominoplasty or breast reduction with the consent of the patients. The tissue donors originate in Asia or were of Caucasian origin.

The keratinocytes isolated from the tissues were cultivated in Keratinocyte-SFM (Gibco®, Life Technologies GmbH, Darmstadt, Germany). The medium was enriched by 5 mg/L of human recombinant epidermal growth factor (EGF), 50 mg/L pituitarium which is an extract from pituitary gland from bovine (Bovine Pituitary Extract, BPE), 1% L-glutamine, 1% streptomycin/amphotericin B (Invitrogen, Karlsruhe, Germany). The cultivation was done at humid atmosphere in the presence of 5% CO₂.

Subconfluent keratinocytes were transferred to sample vessels having 12 sample wells (12-well plates), and were each cultivated in the sample wells. Treatment with the model particles was also done in the sample wells.

### Example 3: Treatment of Cell Cultures with Model Particles

The cultivated keratinocytes (Example 2) were subsequently incubated for 24 hours in the media of example 2 lacking BPE and EFG in the presence of compatible solute S-ectoine having a final concentration of 2 mM.

After incubation with S-ectoine, the suspended and ultrasonically treated model particles (see example 1) were immediately added to the keratinocytes. After an incubation time of 24 hours in the presence of the model particles, RNA was isolated from the keratinocytes and relative RNA content was determined.

### Example 4: Gene Markers

The expression of proinflammatory gene ICAM-1, POMC gene encoding for proopiomelanocortin, MMP1 gene encoding for a human collagenase, and Cyp1a1 encoding for a phase-I-enzyme of Cytochrome P450 superfamily was investigated as a marker for the influence of the model particles on skin condition.

Proopiomelanocortin (POMC) is a precursor protein made of 241 amino acid residues. It is synthesized from pre-proopiomelanocortin (pre-POMC) consisting of a polypeptide being 285 amino acid in length, wherein a signaling peptide made of 44 amino acids is cleaved off during translation. POMC in turn can enzymatically be cleaved of in peptides, such as ACTH, α-MSH, β-MSH, and γ-MSH. The afore-mentioned peptides are peptide hormones and are summarized under the group of melanotropins or hormones stimulating melanocytes (MSH). One exception is ACTH which ranks among the group of melanocortins and is referred to as adrenocorticotropin. Melanotropins regulate the melanin synthesis in pigment forming melanocytes, as well as melanocytes expansion and pigment dispersion, thus having significant influence on pigmentation of the skin. Increased expression of POMC thus indicates increased melanotropin content and is thus an indicator for increased pigmentation of the skin.

The enzyme encoded by Cyp1a1 gene ranks among the group of phase-I-P450-Cytochrome enzymes and mediates metabolism of drugs, foreign substances and environmental toxins. The Cy1a1 enzyme is a monooxygenase and responsible for metabolizing the afore-mentioned compounds. The protein is encoded in endoplasmic reticulum and its expression can severely be induced by polycyclic aromatic hydrocarbons. The enzyme encoded by Cyp1a1 metabolizes environmental noxae, in particular polycyclic aromatic hydrocarbons, into cancer-causing intermediates. Cyp1a2 is a family member related to Cyp1a1. Increased Cyp1a1 expression, and, in particular, increased Cyp1a2 expression, thus proves detoxification reaction.

The collagenase encoded by MMP1 gene ranks among the matrix metalloproteases and cleaves the peptide bond between proline and other amino acids of collagen I, II, III, VII and X, thereby degrading the collagen. Collagenases and, in particular, the MMP1 collagenase are involved in tissue remodeling and biological processes of the skin, such ach morphogenesis or tumor growth (benign or malign tumor formation). Thus, increased expression of MMP1 gene is a proof for increased activity of the skin cells, in particular keratinocytes. Increased MMP1 gene expression can be an indication of excessive cell growth and, thereby, of benign or malign carcinogenesis.

### Example 5: RNA Isolation and PCR

The total RNA content was determined according to Grether-Bech et al. (Grether-Beck S, Mühlberg K, Brenden H, Felsner I, Brynjólfsdóttir A, Einarsson S, Krutmann J. “Bioactive molecules from the Blue Lagoon: in vitro and in vivo assessment of silica mud and microalgae extracts for their effects on skin barrier function and prevention of skin ageing”. Exp Dermatol 2008 17:771.).

For this purpose, the total RNA was isolated using RNeasy Total RNA Kits (Qiagen, Hilden; Germany). The RNA concentration was photometrically determined at 260/280 nm (Biophotometer, Eppendorf, Hamburg, Germany). Aliquots of total RNA of 100 ng were used for first strand cDNA synthesis using Superscript™ III for the transcription step using random primers (Invitrogen, Karlsruhe, Germany).

A specific, complementary primer was designed for each gene, Cyp1a1, MMP1 and POMC, based on the cDNA sequence using Primer Express™ 2.0 software. For the total RNA content a specific primer for the proof of 18s rRNA was designed.

Independent experiments were performed with a triple determination in each case, and the mean was determined. PCR reaction was done using CFX384 Touch™ Real-Time PCR Detection System (Bio-Rad, München, Germany). In order to fluorescent marking SybrGreen-Mix was used (Thermo Fisher Scientific, St. Leon-Rot, Germany). For each experiment three samples, respectively, were determined in duplicate, and the mean was determined.

The PCR protocol comprised 46 cycles, each cycle 15 minutes at 94° C. (activation of heat-activating taq polymerase) 10 seconds at 95° C. (denaturation), 20 seconds at 55° C. (annealing), 20 seconds at 72° C. (elongation).

Likewise, for comparison of relative expression the expression of untreated keratinocytes was investigated. The expression of untreated keratinocytes was set to 1 and the changes in expression were determined according to Livak and Schmittgen of 2001 (Livak K J, Schmittgen TD (2001) “Analysis of relative gene expression data using real-time quantitative PCR and the 2(-Delta Delta C(T)) Method”. Methods 25: 402-408).

### Example 6: Statistics

One-way analysis of variance was used as non-parametric test for comparison of the differences between the measurements, and a p-value of less than 0.05 was specified as being statistically significant (SigmaPlot 12.5). The data underlying the statistical analysis are not shown here.

### Example 7: Protective Effect of a Solute According to the Invention, Here S-Ectoine, on the Skin

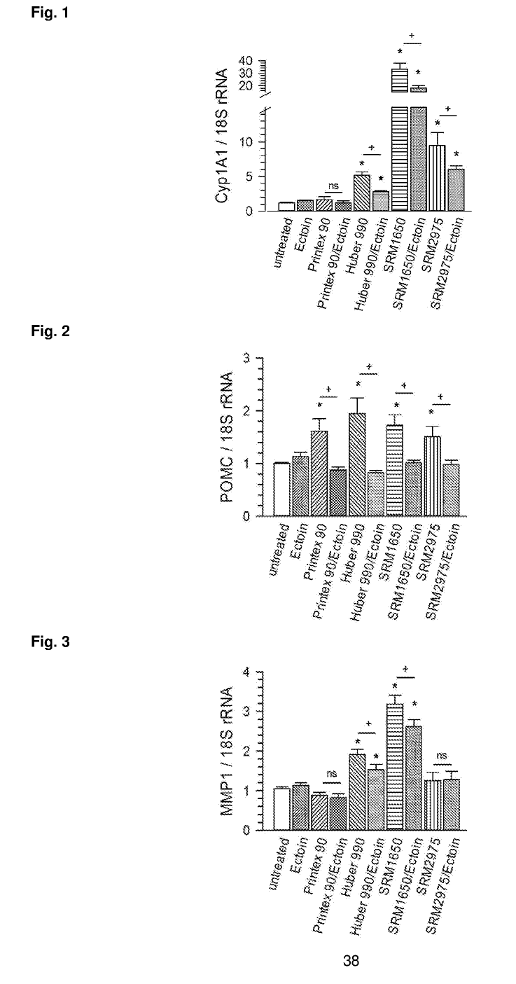

In the following table 3, the effects of all experiments are summarized, as also shown in FIGS. 1, 2, and 3.

| | Expression | ±S-ectoine | Remaining | inhibition |
|---|---|---|---|---|
| | mean ± SD | mean ± SD | activity % | % |
| | Cyp1a1 | | | |
| Printex90 | 1.64 ± 0.43 | 1.19 ± 0.29 | 30 | 70 |
| Huber990 | 5.17 ± 48* | 2.80 ± 0.14⁺ | 43 | 57 |
| SRM1650 | 33.20 ± 4.64* | 18.26 ± 1.72⁺ | 54 | 46 |
| SRM2975 | 9.46 ± 1.90* | 6.03 ± 0.52⁺ | 59 | 41 |
| | POMC | | | |
| Printex90 | 1.61 ± 0.23* | 0.88 ± 0.05⁺ | 0 | 100 |
| Huber990 | 1.94 ± 0.30* | 0.83 ± 0.04⁺ | 0 | 100 |
| SRM1650 | 1.72 ± 0.20* | 1.01 ± 0.05⁺ | 1 | 99 |
| SRM2975 | 1.51 ± 0.20* | 0.99 ± 0.08⁺ | 0 | 100 |
| | MMP1 | | | |
| Printex90 | 0.88 ± 0.08 | 0.82 ± 0.10 | — | — |
| Huber990 | 1.91 ± 0.13* | 1.53 ± 0.13⁺ | 58 | 42 |
| SRM1650 | 3.19 ± 0.22* | 2.62 ± 0.17⁺ | 74 | 26 |
| SRM2975 | 1.27 ± 0.21 | 1.28 ± 0.22 | — | — |

Ultra-fine Printex90 particles enhance the expression of Cyp1a1 mRNA by a factor of 1.64 (mean±0.43) (see table 3, column 2 “Expression”), which is reduced by adding S-ectoine by a factor of 1.19 (mean±0.29).

When treated with Huber990 particles that enhance Cyp1a1 expression by a factor of 5.17, the effect is significantly stronger. It has surprisingly been found that pretreating with S-ectoine results in significant reduction of Cyp1a1 expression by a factor of 2.80. This shows the preventive effect of S-ectoine in prevention of the formation of cancer-causing intermediates from environmental noxae.

Experiments with polycyclic aromatic hydrocarbons, such as SRM1650 and SRM2975, show an even stronger effect. Both particles can be converted into cancer-causing intermediates by Cyp1a1 mediated metabolizing, which may cause skin diseases right up to skin cancer, as described above.

SRM2975 particles enhance Cyp1a1 expression by a factor of 9.46. Pretreating with a solute according to the invention of formula I or of formula II, here S-ectoine, results in inhibition of Cyp1a1 expression by a factor of 6.03.

SRM1650 particles enhance Cyp1a1 expression by a factor of 33.20. Pretreating with a solute according to the invention of formula I or of formula II, here S-ectoine, results in significant inhibition of Cyp1a1 expression by a factor of 18.25. This proves the protective effect of the solutes according to the invention of formula I and of formula II in the prevention of pathologic efflorescences induced by polycyclic aromatic hydrocarbons, in particular of those comprising an alteration of keratinocytes.

All of the tested particles Printex90, Huber990, SRM1650, as well as SRM2975 resulted in significantly enhanced POMC expression after incubation of keratinocytes in the presence of the afore-mentioned particles for 24 hours.

S-ectoine pretreatment for 24 hours resulted in significant inhibition of POMC expression, nearly to the initial level of untreated keratinocytes, for all of the tested germs, as summarized in table 3.

The protective and inhibitory effect of S-ectoine on POMC expression induced by airborne particles proves the effectiveness of the solutes according to the invention of formula I and of formula II in the prevention or treatment of cosmetic or pathologic efflorescences, which exhibit phenotypic alteration in pigmentation, as well as against skin ageing.

Due to reduced POMC expression less proopiomelanocortin is synthesized. Consequently, fewer melanotropins are cleaved off, thereby in turn stimulating fewer pigment forming cells, melanocytes. This result in reduced pigment formation (melanin) compared to untreated cells exposed to airborne particles. Phenotypically, for example, hypopigmentation, hyperpigmentation, cosmetic alteration in pigmentation and senile atrophy, micro- and macroscopically visible, are thus reduced by pretreatment with the solute according to the invention of formula I and of formula II, or the solute mixture.

Similar effects could be shown in the proof of inhibition of the solute according to the invention, here S-ectoine, on MMP1 expression induced by Huber990 and SRM1650. Inhibition of the expression of MMP1 gene proves downregulation of collagenase, hence reduced collagen degradation, and thus an inhibitory effect against skin ageing. In this way the solute according to the invention of formula I and of formula II, as well as the mixture, preferably comprising S-ectoine and (S,S)-hydroxyectoine, shows positive effect in the prevention or treatment of skin ageing, senescence of the skin or elasticity displacement, caused by airborne particles.

1. A method for preventing or treating cosmetic or pathologic efflorescences in a subject caused by airborne particles, the method comprising administering to the subject an effective amount of a composition comprising at least one compound selected from the group consisting of compounds of formula I, compounds of formula II, physiologically compatible salts thereof, stereoisomeric forms thereof, physiologically compatible salts of the stereoisomeric forms thereof, and combinations thereof, 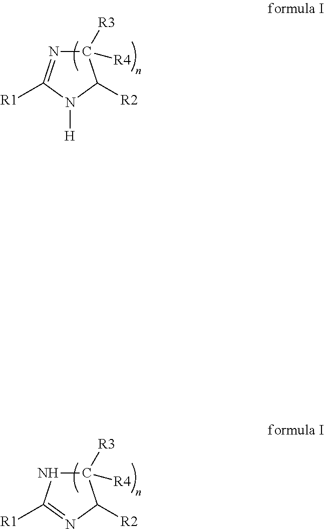
wherein:
R1 is H or C₁-C₄ alkyl;
R2 is H, COOH, COO—[C₁-C₄ alkyl], or CO—NH—R5;
R3 and R4 are each independently H or OH;
R5 is H, C₁-C₄ alkyl, an amino acid residue, a dipeptide residue, or a tripeptide residue; and
n is 1, 2 or 3,
thereby preventing or treating cosmetic or pathologic efflorescences in the subject.

2. The method according to claim 1, wherein the at least one compound is selected from the group consisting of S-ectoine, R-ectoine, (S,S)-hydroxyectoine, (S,R)-hydroxyectoine, (R,S)-hydroxyectoine, (R,R)-hydroxyectoine S-homoectoine, physiologically compatible salts thereof, amides thereof, esters thereof, and combinations thereof.

3. The method according to claim 1, wherein the at least one compound is present in enantiopure form with a purity of greater than or equal to 90%.

4. The method according to claim 1, wherein the at least one compound or the solute mixture is an expression inhibitor of genes associated with efflorescences.

5. The method according to claim 4, wherein the at least one compound or the solute mixture inhibits the expression of a gene selected from the group consisting of POMC, Cyp1a1, and MMP1, which is induced by airborne particles.

6. The method according to claim 1, wherein the cosmetic or pathologic efflorescence is an efflorescence of melanocytes and/or keratinocytes.

7. The method according to claim 1, wherein the cosmetic or pathologic efflorescence comprises an alteration of at least one cell layer in at least one part of the skin, being phenotypically visible on the skin surface,

8. The method according to claim 1, wherein the solute mixture comprises at least two compounds selected from the group consisting of compounds of formula I, compounds of formula II, physiologically compatible salts thereof, stereoisomeric forms thereof, physiologically compatible salts of the stereoisomeric forms thereof, and combinations thereof.

9. The method according to claim 1, wherein the solute mixture comprises, based on the sum of all compounds having a total content of 100% by weight, greater than or equal to 85% by weight S-ectoine, and less than or equal to 15% by weight (S,S)-hydroxyectoine.

10. The method according to claim 1, wherein the airborne particles that cause the cosmetic or pathologic efflorescences comprise particulate and/or fibrous environmental noxae according to specification of DIN EN 481, WHO/SDE/OEH/99.14 or ISO 4225.

11. The method according to claim 1, wherein the airborne particles comprise nanoparticles, carbon nanoparticles, particles containing polycyclic aromatic hydrocarbons, and/or diesel soot particles.

12. A composition comprising at least one compatible solute or one solute mixture comprising at least two compatible solutes for use in the prevention or treatment of cosmetic or pathologic efflorescences caused by airborne particles, wherein the compatible solute or the solute mixture comprises at least one compound selected from the group consisting of compounds of formula I, compounds of formula II, physiologically compatible salts thereof, stereoisomeric forms thereof, physiologically compatible salts of the stereoisomeric forms thereof, and combinations thereof, 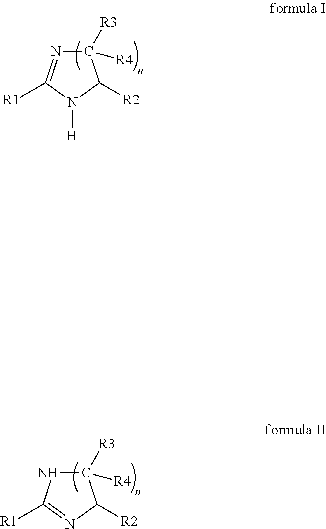
and further wherein:
R1 is H or C₁-C₄ alkyl;
R2 is H, COOH, COO—[C₁-C₄ alkyl], or CO—NH—R5;
R3 and R4 are each independently H or OH;
R5 is H, C₁-C₄ alkyl, an amino acid residue, a dipeptide residue, or a tripeptide residue; and
n is 1, 2 or 3.

13. The composition according to claim 12, wherein the at least one compatible solute or the solute mixture is present in the composition in an amount of 0.0001% by weight to 50% by weight based on the total content of the composition.

14. The composition according to claim 12, wherein the at least one compatible solute or the solute mixture is present in the composition in an amount of 0.0001% by weight to 10% by weight based on the total content of the composition.

15. The composition according to claim 12, wherein the airborne particles that cause the cosmetic or pathologic efflorescences comprise particulate and/or fibrous environmental noxae according to specification of DIN EN 481, WHO/SDE/OEH/99.14 or ISO 4225.

16. The composition according to claim 12, wherein the composition is present in a form selected from the group consisting of:
i) solid forms comprising one or more powders, tablets, granules, film-coated tablets, dragees, capsules, effervescent tablets, powders, soaps, and combinations thereof;
ii) liquid forms comprising one or more solutions, injections, infusions, tinctures, infusion solutions, suspensions, syrups, juices, emulsions, applications, foams, creams, lotions, surfactant-containing cleaning preparations, oils, and combinations thereof and
iii) mixtures comprising one or more sprays, aerosols, inhalants, ointments, pastes, and combinations thereof.

17. The composition according to claim 12, wherein the cosmetic efflorescence is a senescence of skin exposed to airborne particles, and further wherein the skin exhibits deviating pigmentation, deviating expression of at least one gene, and/or deviating production of at least one biomolecule as compared to skin that has not been exposed to the airborne particles.

18. The composition according to claim 12, wherein the cosmetic efflorescence caused by airborne particles is a skin aging and/or pigmentation.

19. The composition according to claim 12, wherein the pathologic efflorescence comprises depigmentation, hypopigmentation, hyperpigmentation, acquired melanotic and non-melanotic hyperpigmentation, incontinentia pigmenti, dermatoses, dermatoses caused by building materials or cement, pigmented spots, anuli pigmentosi, prurigo pigmentosa, peribuccal pigmentation, carcinomas, spinocellular coarcinoma, pigmented spindle-cell tumor, skin diseases caused by airborne particles containing polycyclic aromatic hydrocarbons, benign or malign tumor formation, Leschke syndrome, skin metastases, Becker's nevus, malignant melanoma, pigmented neurofibroma, urticarial pigmentosa, Peutz-Jeghers syndrome, epidermodysplasia verruciformis, tacrolimus, pimecrolimus, vitiligo, atrophy-associated diseases comprising exsiccation eczemas, elastoses, purpura senilis, lentigo solaris, or Favre-Racouchot syndrome.
